(19) United States
(12) Patent Application Publication
(10) Pub. No.: US 2011/0071487 A1
(43) Pub. Date: Mar. 24, 2011

(54) Shape modification of absorbent portion and crotch area of adult male disposable undergarments to improve comfort in adult males suffering only minor incontinence while reducing cost to manufacture

(76) Inventor: Thomas Joseph Sabiston, JR., Panama City, FL (US)

(21) Appl. No.: 12/925,667

(22) Filed: Oct. 27, 2010

(51) Int. Cl. A61F 13/491; A61F 13/49

(52) U.S. Cl. 604/385.01

(57) ABSTRACT

This patent is for the protection of a proposed redesign of disposable undergarments for adults and even children, to correct years of design changes that have basically encompassed only changes in manufacturing process, or materials in order to utilize developing technology for improved performance. This proposed design is primarily for a shape change that results in improved comfort and reduced unpleasant side effects for the wearer, and lower costs in manufacturing.

This invention is a modification or re-design of presently available male disposable undergarments. This re-design basically creates an alternate new product for selection by males suffering minor incontinence. It offers a more comfortable fit and should cost less to manufacture due to a reduction of required absorbent materials.

### CROSS REFERENCE TO RELATED APPLICATIONS

Not Applicable

### STATEMENT REGARDING FEDERALLY SPONSORED RESEARCH OR DEVELOPMENT Not Applicable

### REFERENCE TO SEQUENCE LISTING, A TABLE, COMPUTER PROGRAM, or CD Not Applicable

### BACKGROUND

This invention came about from one of the inventors having to use the current product for an extended period of time, during which he was confronted with increased leg and skin irritation as a result of the present product design. Although several different product name brands were tried, it was noted that they were all basically the same in design, with no major differences between the products for women and men. The inventors decided that patients having to wear this type of product should have a better choice available to meet the specific and varying needs of consumers.

### SUMMARY

This product, in present design, is used by people suffering from varying degrees of incontinence. Yet, other than size, there seems to be very little, if any, difference in the overall design and size of the absorbent padding. Although men and women are certainly different in physical build, close examination of the men's and women's products reveals basically no difference in the material placed in the crotch area. Mothers learned quickly with children's disposable diapers (the forerunners to the adult disposable undergarments), that boys wet the front of a diaper higher up than girls do. In the adult world, men are simply not used to having a large wad of material compressed between their legs, and packed into their crotch all day long. Yet the width of the absorbent padding material in current male disposable garments can be as much as 4-inches across and ¼″ thick. Obviously, men not used to having that much material folded and compressed in this region of the body, can suffer comfort issues as well as side effects that can progress into serious physical irritation.

One major reason for male incontinence, besides age, is the result of having to have a radical prostectomy due to cancer. More than 50,000 adult males have this surgery every year. After the initial recovery period, mild incontinence can last for a year or more. During this prolonged period, these men require only minimal protection , but are being forced to wear garments designed for maximum protection because no other viable products of lesser design are presently available. In reviewing more than 300 patents issued as far back as 1978, the providers of these products have been able to make many improvements for moisture absorption and the ability to retain moisture in a dry or gel-like state, due to technological advances in absorptive materials. Yet, during that same period, there have been no changes (or efforts) toward improving comfort, or to accommodate those with only a minor incontinence problem. This proposed re-design of the product directly addresses, and creates, a solution to that issue. Hopefully this change will increase the comfort and lack of irritation for millions of men who suffer from only minor urinary incontinence. Naturally, the granting of a patent does not in any way guarantee current manufacturers will adopt this product re-design. However, it will protect the inventor against them doing so and not recognizing the inventor for their past failure to recognize and offer such a change or alternative to male consumers for so many years.

An additional benefit to this proposed re-design should be a reduction in cost to the manufacturer with the modified product due to a noticeable reduction in absorptive material needed to complete the alternative garment.

### DESCRIPTION OF DRAWING VIEWS:

FIG. 1 is a dimensional view of, and representative of, currently available products. For the sake of clarity, we will refer to the overall garment as a “brief”. The brief is the paper underwear like structure, that contains an absorptive pad, we will refer to only as the “pad”and is designated with the number “1”. FIG. 2 shows the brief design, laid flat, with the front of the garment at the top and the back of the garment at the bottom with the pad (1) situated in the center. The edges (2) in FIG. 2 will eventually be brought together and joined to each other to form the finished brief. FIG. 3 shows a typical absorptive pad (1), which is inserted in the center of the brief as indicated in the drawings. FIG. 4 is a frontal view of the finished brief product, which is arrived at by bringing the top and bottom of FIG. 2 together and joining and tacking the sides together as indicated by the number “3”. With the absorptive pad in place, and the front and back joined at the sides, the result is the finished brief garment illustrated by FIG. 5. FIGS. 2-5 illustrate currently available product. The following numbers relate to the same items in all graphics:

#1 Absorptive Pad

#2 Brief sides before joining

#3 Brief sides after joining

FIG. 6 illustrates the typical present brief design and FIG. 7 is the proposed new design for the brief. FIG. 8 shows present absorption pad design and FIG. 9 shows proposed new design for the absorption pads. Note the reduction in size and use of material in the crotch area. The suggested reduction shall be from the typically current 4-inch wide pads to 1-inch or less, while leaving the front and rear pads the same width at both ends for front and back placement.

FIG. 10 shows a current typical front view of the brief, after the edges have been joined, with the absorption pad in place. FIG. 11 shows a proposed frontal view of the brief with the modified crotch and modified pad in place. FIG. 12 is a dimensional rendering of a current finished product before modification and FIG. 11 is the same product after modification.

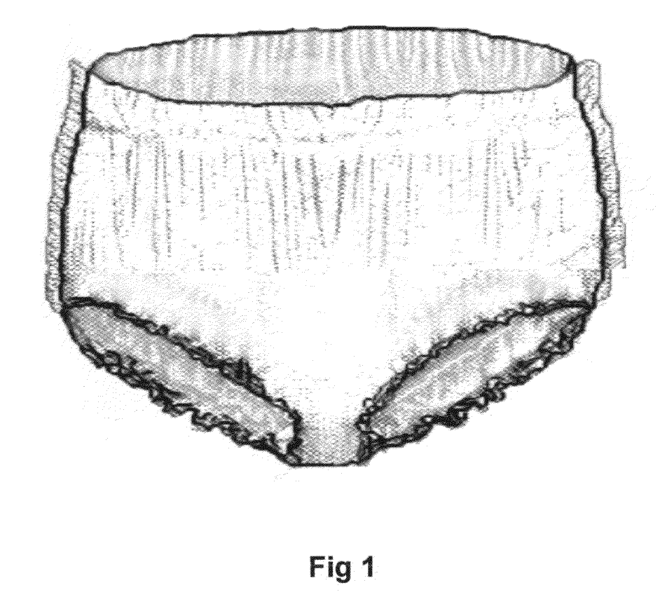

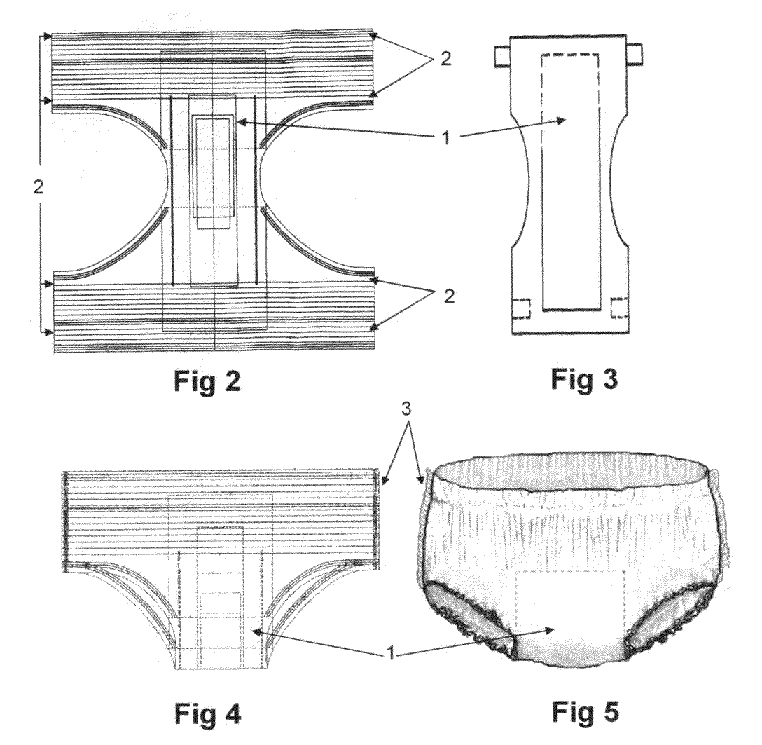

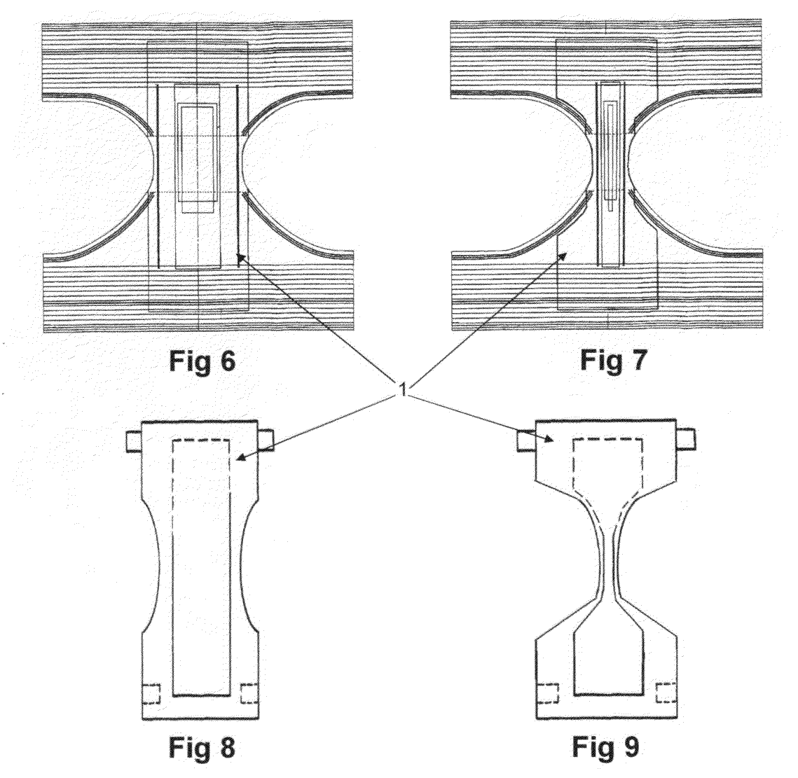

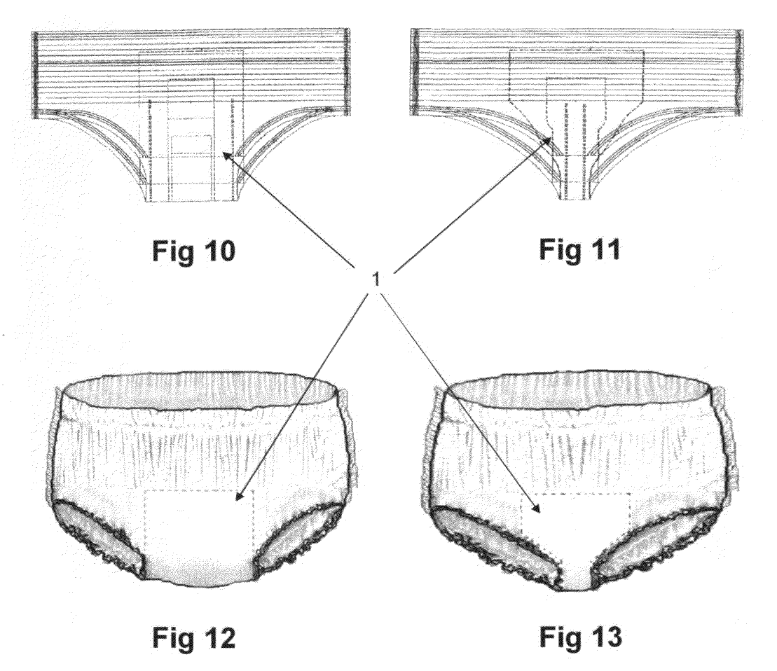

As mentioned earlier, we reviewed more than 300 patents as far back as 1978. Some of the earlier absorption pads were very complex in their design and construction in order to provide a decent level of protection. The newer pads are far more simple and contain mostly absorptive fibers and crystals that make accommodating the proposed design changes even easier.

It is important to note that this application for patent does not change any technical specifications in current existing products, manufacturing processes, or composition of materials. We wish to patent the change in shape for the basic brief and the inserted absorption pad to significantly decrease the width of material in the crotch. An alternative to decreasing the width, would be to totally eliminate the absorption pad through the crotch and have only two pads, one front and one rear, stopping at some determined point before entering the crotch area.

### DETAILED DESCRIPTION

As previously outlined, the design change (shape alteration) that we are asking be patented, is simple in nature, but important to those who have to wear the product, and it has been ignored for more than a quarter century of previous product developments and alterations.

1. We claim protection for any design changes that reduce the size, bulk or shape to the crotch area of previous and currently designed male disposable undergarments as herein indicated for both the brief and absorption pad.

2. We claim protection for any design changes that reduce the size, bulk or shape to the crotch area of male disposable undergarments through the total elimination of any absorption material within the crotch area and combined with the use of two independent absorption pads located front and back of the brief.

3. We claim the protection for the application of these same design modifications if made to similar disposable garments made for females and/or children of either sex.
